(19) United States
(12) Patent Application Publication
(10) Pub. No.: US 2011/0217258 A1
(43) Pub. Date: Sep. 8, 2011

(54) Combined Use of Bendamustine, Doxorubicin and Bortezomib for the Treatment of Multiple Myeloma

(75) Inventor: Sherif S. Farag, Carmel, IN (US)

(73) Assignee: Indiana University Research and Technology Corporation, Indianapolis, IN (US)

(21) Appl. No.: 13/096,300

(22) Filed: Apr. 28, 2011

Related U.S. Application Data
(63) Continuation of application No. PCT/US2009/064009, filed on Nov. 11, 2009.
(60) Provisional application No. 61/114,284, filed on Nov. 13, 2008.

(51) Int. Cl. A61K 31/704; A61K 38/19; A61P 35/00

(52) U.S. Cl. 424/85.1; 514/34

(57) ABSTRACT

Methods for treating multiple myeloma by administering a combination of a proteasome inhibitor, bendamustine and doxorubicin are described.

### TECHNICAL FIELD

Combination drug therapy for multiple myeloma.

### BACKGROUND

Multiple myeloma (MM) is a malignancy of the bone marrow in which cancerous plasma cells grow out of control and create a tumor. When these tumors grow in multiple sites, they are referred to as multiple myeloma. Normally, plasma cells make up less than five percent of the cells in bone marrow, but people with multiple myeloma have anywhere from ten percent to more than ninety percent. MM is also characterized by overproduction of intact monoclonal immunoglobulins (IgG, IgA, IgD, or IgE, “M-proteins”) or Benece-Jones protein (free monoclonal light chains). Hypercalcemia, anemia, renal damage, increased susceptibility to bacterial infection, and impaired production of normal immunoglobulin are common clinical manifestations of MM. The overgrowth of malignant plasma cells in bone marrow can cause a number of serious problems throughout the body. Over time, the abnormal cells can permeate the interior of the bone and erode the bone cortex (outer layer). These weakened bones are more susceptible to bone fractures, especially in the spine, skull, ribs, and pelvis.

The annual incidence of MM is approximately 4 per 100,000 people, and the condition is particularly common in the elderly population with a median age of 65 years; only 3% of patients with MM are less than 40 years old. Although patients may initially respond to chemotherapy and/or steroids, most ultimately suffer from resistant disease. Ma et al.(2003) “The proteasome inhibitor PS-341 markedly enhances sensitivity of multiple myeloma tumor cells to chemotherapeutic agents.” Clinical Cancer Research 9:1136-1144. Cytotoxic chemotherapy prolongs the survival of symptomatic patients. However, the prognosis of treated patients with MM remains poor. Thus, treatment for MM is essentially palliative. While high-dose chemotherapy with stem cell support increases the proportion of complete responses, the effect on prolongation of overall survival remains relatively modest, and all patients invariably relapse. Attal et al. (1996) “A prospective, randomized trial of autologous bone marrow transplantation and chemotherapy in multiple myeloma”. Intergroupe Francais du Myelome. N Engl J Med. 335:91-97. See also, Child et al. (2003) “High-dose chemotherapy with hematopoietic stem-cell rescue for multiple myeloma.” N Engl J Med. 348:1875-1883. Intensifying treatment with tandem cycles of high-dose alkylating agents (with or without total body radiation) have improved outcome in some studies. Attal et al. (2003) “Single versus double autologous stem-cell transplantation for multiple myeloma.” N Engl J Med. 2003; 349:2495-2502. See also, Barlogie et al. (2004) “Treatment of multiple myeloma.” Blood 103: 30-32. However, improvement was not seen in all studies. Cavo et al. (2003) “Single versus tandem autologous transplants in multiple myeloma: Italian experience.” The Hematology Journal. 2003:4 (suppl). For patients who progress after primary chemotherapy, including autologous stem cell transplantation, further chemotherapy is generally of limited benefit. Overall, the results of conventional cytotoxic chemotherapy in MM suggest that a plateau is usually reached, indicating the need for novel therapies.

Proteasome inhibitors have shown promise in the treatment of MM. The proteasome (also referred to as multicatalytic protease (MCP), multicatalytic proteinase, multicatalytic proteinase complex, multicatalytic endopeptidase complex, 20S, 26S, or ingensin) is a large, multiprotein complex present in both the cytoplasm and the nucleus of all eukaryotic cells. It is a highly conserved cellular structure that is responsible for the ATP-dependent proteolysis of most cellular proteins (Tanaka, Biochem Biophy. Res. Commun., 1998, 247, 537). The 26S proteasome consists of a 20S core catalytic complex that is capped at each end by a 19S regulatory subunit. The 26S proteasome is able to degrade proteins that have been marked by the addition of ubiquitin molecules. It is believed that proteasome inhibitors, which inhibit proteasome activity, can arrest or delay cancer progression by interfering with the ordered degradation of cell cycle proteins or tumor suppressors.

Bortezomib, also known as PS-341 or [(1R)-3-methyl-1-({(2S)-3-phenyl-2-[(pyrazin-2-ylcarbonyl)amino]propanoyl}amino)butyl]boronic acid, is a boronic acid dipeptide proteasome inhibitor that has shown anti-tumor activity both in vitro and in clinical trials involving MM patients. Mitsiades et al. (2003) “The proteasome inhibitor PS-341 potentiates sensitivity of multiple myeloma cells to conventional chemotherapeutic agents: therapeutic applications.” Blood. 101:2377-2380. Field-Smith et al. (2006) “Bortezomib (Velcade™) in the treatment of multiple myeloma.” Therapeutics and Clinical Risk Management. 2(3):271-279. Bortezomib inhibits the activation of the transcription factor NF-κB, induces apoptosis, inhibits MM cell binding to bone marrow stromal cells and inhibits production of MM growth and survival factors in the BM milieu. It is currently approved for the treatment of MM in the relapsed setting after transplantation, or as a second line treatment in patients unsuitable for transplantation.

In addition to bortezomib, other proteasome inhibitors are also known. For example, US 2005/0107307 A1 describes a group of novel boronic acid proteasome inhibitors, including the compound known as CEP-18770. CEP-18770, whose chemical name is [(1R)-1-[[(2S,3R)-3-hydroxy-2-[6-phenyl-pyridine-2-carbonyl)amino]-1-oxobutyl]amino]-3-methylbutylboronic acid, has been shown to be orally active and have a favorable tumor selectivity profile for the treatment of MM and other malignancies responsive to proteasome inhibition. Piva, et al. (2008) “CEP-18770: A novel orally active proteasome inhibitor with a tumor-selective pharmacologic profile competitive with bortezomib.” Blood; 111:2765-2775.

The combination of bortezomib and DOXIL® pegylated liposomal doxorubicin has been shown to be an effective regimen in MM, even in patients with relapsed and refractory disease. Orlowski et al. (2007) “Randomized Phase III study of pegylated doxorubicin plus bortezomib compared with bortezomib alone in relapsed or refractory multiple myeloma: combination therapy improves time to progression.” J Clin Oncol 25:3892-901. Patients with relapsed/refractory disease are defined as those who, having achieved minor response or better, relapse and then progress while on salvage therapy, or experience progression within sixty days of their last therapy.

The FDA has approved use of a combination of pegylated liposomal doxorubicin (PLD) and bortezomib for the treatment of multiple myeloma patients that have received at least one prior therapy and have not previously received bortezomib. Richardson et al. (2007) “The treatment of relapsed and refractory multiple myeloma” Hematology 1:317-323. In the approved regimen, bortezomib is administered at a dose of 1.3 mg/m² as intravenous bolus on days 1, 4, 8 and 11 of every three weeks. VELCADE (bortezomib) is supplied as a powder that is made up with saline to 1 mg/ml. Thus, patients typically are administered about 2-2.5 ml per dose as an iv bolus. DOXIL® PLD (liposomal doxorubicin) is administered at a dose of 30 mg/m². The DOXIL® PLD preparation is diluted with 5% dextrose to 250 ml and administered as a 1-hr intravenous infusion on day 4 following bortezomib. With the first DOXIL® PLD dose, an initial rate of 1 mg/min should be used to minimize the risk of infusion-related reactions. If no infusion-related adverse reactions are observed, the infusion rate should be increased to complete the administration of the drug over one hour. Patients may be treated for up to 8 cycles until disease progression or the occurrence of unacceptable toxicity.

Bendamustine is a purine analogue/alkylator hybrid that has shown potent clinical activity against several human cancers, including non-Hodgkin's lymphoma (NHL) and multiple myeloma (MM). The chemical name of bendamustine hydrochloride is 1H-benzimidazole-2-butanoic acid, 5-[bis(2-chloroethyl)amino]-1-methyl-, monohydrochloride. Bendamustine hydrochloride has the following structural formula:

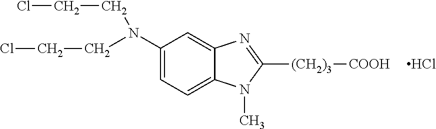

Bendamustine has been shown to be clinically efficacious in non-Hodgkin's lymphoma, CLL, breast cancer, and MM. In combination with corticosteroids, bendamustine was shown to be superior to melphalan in newly diagnosed patients with MM. Ponisch et al. (2006) J Cancer Res Clin Oncol 132: 205-12.

The combination of bendamustine, dexamethasone and bortezomib was shown to be effective in the treatment of multiple myeloma. Fenk et al., “Escalation therapy with bortezomib, dexamathasone and bendamustine for patients with relapsed or refractory multiple myeloma”; Leuk Lymphoma (2007) December; 48:12, 2298-9. The combination of bendamustine, bortezomib and prednisone was also shown to achieve a response rate of 80%. Lonial S., “Multiple myeloma: novel approaches for relapsed disease”; Clin Lymphoma Myeloma (2007) December; 8: Suppl 1, S18-23.

Published data has shown, however, that the combination of bendamustine with doxorubicin (or mitoxantrone) is antagonistic in lymphoma cells in vitro, discouraging the investigation of bendamustine/doxorubicin combination therapy in MM. Chow et al. (2001) “In vitro induction of apoptosis of neoplastic cells in low-grade non-Hodgkin's lymphomas using combinations of established cytotoxic drugs with bendamustine.” Haematologica 86:485-93.

There remains a need for treatment options that can offer the best long-term outcome for multiple-myeloma patients. The need is especially urgent for novel therapies for patients with relapsed or refractory disease who are typically more symptomatic and may be older with potential comorbidities and are especially challenging to treat. Until the study disclosed herein, the triple combination therapy of a proteasome inhibitor, bendamustine and doxorubicin had never been investigated. This triple combination therapy offers a solution to the treatment of both recently diagnosed MM patients and patients with relapsed or refractory disease.

All references cited are hereby incorporated by reference.

### SUMMARY

Provided are methods for treating multiple myeloma in a subject. The subject is administered an effective amount of a combination comprising a proteasome inhibitor, bendamustine and doxorubicin.

In certain preferred embodiments, the proteasome inhibitor is bortezomib.

In other preferred embodiments, the proteasome inhibitor is [(1R)-1-[[(2S,3R)-3-hydroxy-2-[6-phenyl-pyridine-2-carbonyl)amino]-1-oxobutyl]amino]-3-methylbutylboronic acid.

### BRIEF DESCRIPTION OF THE DRAWINGS

FIG. 1: Representative experiment showing synergistic cytotoxic activity of bendamustine when combined with doxorubicin and bortezomib in H929 cells. B, 15 μg/ml bendamustine; D, 0.03 μg/ml doxorubicin; V, 2.5 nmol/L bortezomib; B+D, 15 μg/ml bendamustine and 0.03 μg/ml doxorubicin; B+V, 15μg/ml bendamustine and 2.5 nmol/L bortezomib; D+V, 0.03 μg/ml doxorubicin and 2.5 nmol/L bortezomib; B+D+V, the combination of 15 μg/ml bendamustine with 0.03 μg/ml doxorubicin and 2.5 nmol/L bortezomib. Synergy of the drug combination was defined when the measured cell survival was lower than the product of the effect of each single drug.

### DETAILED DESCRIPTION OF ILLUSTRATIVE EMBODIMENTS

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “subject” includes warmblooded animals, preferably mammals, including humans. In a preferred embodiment, the subject is a primate. In an even more preferred embodiment, the subject is a human.

I. Methods of the Invention

Provided are methods for treating multiple myeloma in a subject. The subject is administered an effective amount of a combination comprising a proteasome inhibitor, bendamustine and doxorubicin. Surprisingly, the combination is synergistic, notwithstanding the fact that the combination of bendamustine and doxorubicin is known to be antagonistic in lymphoma cells. Chow et al. (2001) “In vitro induction of apoptosis of neoplastic cells in low-grade non-Hodgkin's lymphomas using combinations of established cytotoxic drugs with bendamustine.” Haematologica 86:485-93. The methods of the present invention allow for a reduction in the amount of the therapeutic agents required to be effective, which is advantageous since it can result in fewer side effects in the subject being treated.

Any suitable proteasome inhibitor may be used according to the present invention. A number of boronic acid proteasome inhibitors useful in the present invention are described in Adams, et al., U.S. Pat. Nos. 5,780,454, 6,083,903, 6,617,317, 6,747,150 and 7,119,080, and Gupta, U.S. Pat. Nos. 6,713,446, 6,958,319. Preferred among this group of boronic acids is the compound [(1R)-3-methyl-1-({(2S)-3-phenyl-2-[(pyrazin-2-ylcarbonyl)amino]propanoyl}amino) butyl]boronic acid, also known as bortezomib and marketed by Millennium Pharmaceuticals under the tradename Velcade®. Bortezomib has the following structure:

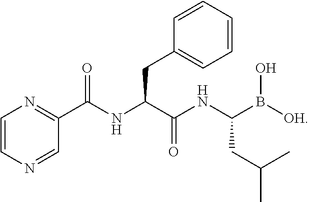

Another group of boronic acid proteasome inhibitors useful in the present invention is described in Bernardini, et al., U.S. Published Application No. US2005/0107307. Preferred among these compounds is [(1R)-1-[[(2S,3R)-3-hydroxy-2-[6-phenyl-pyridine-2-carbonyl)amino]-1-oxobutyl]amino]-3-methylbutylboronic acid, also known as CEP-18770, which has the following structure:

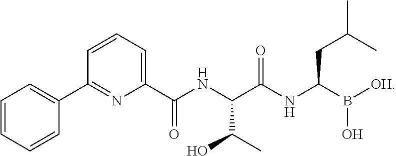

Additional proteasome inhibitors are described in U.S. Pat. No. 7,223,745, and U.S. Published Application No. 2006/0189806 A1, both of which are incorporated herein by reference in their entireties, for all purposes.

Doxorubicin, which is used in the present invention, is an anthracycline antibiotic, closely related to the natural product daunomycin. It may be used in the present invention in any suitable form, including as a salt, ester, polymorph, or solvate. Doxorubicin was first isolated from a mutated strain of Streptomycin and was initially named adriamycin, but its name was later changed to doxorubicin. Arcamone et al. (1969) “Adriamycin, 14-hydroxydaunomycin, a new antitumor antibiotic from S. peucetius var. caesius.” Biotechnol. Bioeng. 11:1101-1110. Doxorubicin consists of a naphthacenequinone nucleus linked through a glycosidic bond at ring atom 7 to an amino sugar, daunosamine. A preferred salt is doxorubicin hydrochloride: 5,12-Naphthacenedione, 10-[(3-amino-2,3,6-trideoxy-α-L-lyxohexopyranosyl)oxy]-7,8,9,10-tetrahydro-6,8,11-trihydroxy-8-(hydroxylacetyl)-1-methoxy-, hydrochloride (8S-cis)-. The structural formula is as follows:

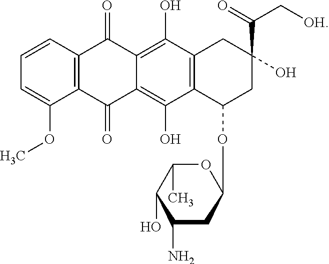

A pegylated liposomal form of doxorubicin (PLD) marketed under the tradename DOXIL® is especially preferred. See, for example, Huang et al, U.S. Pat. No. 4,927,571. DOXIL® PLD is provided as a sterile, translucent, red liposomal dispersion in 10-mL or 30-mL glass, single use vials. Each vial contains 20 mg or 50 mg doxorubicin HCl at a concentration of 2 mg/mL and a pH of 6.5. The STEALTH® liposome carriers are composed of N-(carbonyl-methoxypolyethylene glycol 2000)-1,2-distearoyl-sn-glycero-3-phosphoethanolamine sodium salt (MPEG-DSPE), 3.19 mg/mL; fully hydrogenated soy phosphatidylcholine (HSPC), 9.58 mg/mL; and cholesterol, 3.19 mg/mL. Each mL also contains ammonium sulfate, approximately 2 mg; histidine as a buffer; hydrochloric acid and/or sodium hydroxide for pH control; and sucrose to maintain isotonicity. Greater than 90% of the drug is encapsulated in the STEALTH® liposomes. Liposomes are microscopic vesicles composed of a phospholipid bilayer that are capable of encapsulating active drugs. The STEALTH® liposomes of DOXIL® PLD are formulated with surface-bound methoxypolyethylene glycol (MPEG), a process often referred to as pegylation, to protect liposomes from detection by the mononuclear phagocyte system (MPS) and to increase blood circulation time. It is hypothesized that because of their small size (ca. 100 nm) and persistence in the circulation, the pegylated DOXIL® PLD liposomes are able to penetrate the altered and often compromised vasculature of tumors.

MPEG-DSPE has the following structural formula:

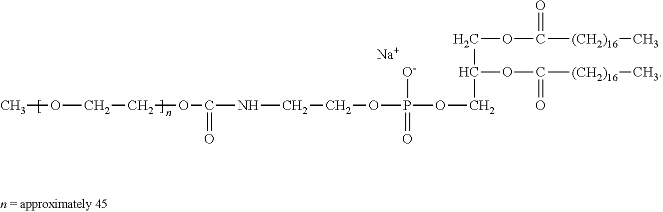

HSPC has the following structural formula:

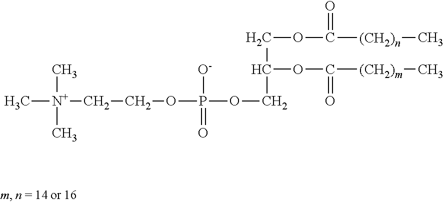

The main benefits of this form of doxorubicin are said to be a reduction in cardiotoxicity. The drug-protective effect of liposomes is due, at least in part, to a marked alteration in tissue disposition and drug-release rate of the injected drug.

Bendamustine may be used in the present invention in the present invention in any suitable form, including as a salt, ester, polymorph, or solvate. A preferred salt form is bendamustine hydrochloride. TREANDA® (bendamustine hydrochloride) for Injection is especially preferred. TREANDA is supplied as a sterile non-pyrogenic white to off-white lyophilized powder in a single-use vial. Each vial contains 100 mg of bendamustine hydrochloride and 170 mg of mannitol, USP. The vial may be reconstituted with 20 mL of Sterile Water for Injection, USP, and may be administered by transferring the required dose to a 500 mL infusion bag of 0.9% Sodium Chloride Injection USP. The pH of the reconstituted solution is preferably 2.5-3.5.

Any suitable method of administration may be used. Examples include injection (subcutaneous, intravenous, parenterally, intraperitoneally, intrathecal, etc.), oral, inhalation, and transdermal. The injection can be bolus injections or can be continuous infusion. Depending on the route of administration, the proteasome inhibitor, bendamustine and/or doxorubicin can be coated with or disposed in a selected material to protect it from natural conditions which may detrimentally affect its ability to perform its intended function. The proteasome inhibitor, bendamustine and doxorubicin can be administered alone, or in conjunction with a pharmaceutically acceptable carrier. Further, the proteasome inhibitor, bendamustine and doxorubicin can be administered individually (i.e., sequentially), together as a mixture, or any combination thereof (e.g., proteasome inhibitor and bendamustine together, doxorubicin individually). The proteasome inhibitor, bendamustine and/or doxorubicin can be administered as a prodrug which is converted to its active form in vivo.

The effective amount of the combination is that amount necessary or sufficient to treat multiple myeloma, e.g. prevent the undesirable cell growth, or reduce the size of a pre-existing benign cell mass or malignant tumor in the subject. The effective amount can vary depending on such factors as the type of cell growth being treated or inhibited, the type of therapeutic agent(s) employed, the particular proteasome inhibitor, the size of the subject, or the severity of the cancer cell growth or tumor. Other factors to be taken into account include the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the proteasome inhibitor can be provided in an aqueous physiological buffer solution containing about 0.1 to about 10% w/v of the compound for parenteral administration. Some typical dose ranges for the proteasome inhibitor are from about 1 μg/kg to about 1 g/kg of body weight per day. In some embodiments, the dose range is from about 0.01 mg/kg to about 100 mg/kg of body weight per day. The dosage for the proteasome inhibitor is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The choice of each of the individual agents (proteasome inhibitor or chemotherapeutic agent) which make up the combination can affect what constitutes an effective amount. One of ordinary skill in the art would be able to study the aforementioned factors and make the determination regarding the effective amount of the combination of the proteasome inhibitor or therapeutic agent without undue experimentation. For example, an in vitro assay can be used to determine an effective amount of the combination of the proteasome inhibitor and a chemotherapeutic agent. The ordinarily skilled artisan would select an appropriate amount of each individual agent in the combination for use in the aforementioned in vitro assay. The cell survival fraction can be used to determine whether the selected amounts were an effective amount for the particular combination of agents. For example, the selected amounts used within the assay preferably should result in a killing of at least 50% of the cells, more preferably 75%, and most preferably at least 95%. In a preferred embodiment, the effective dose of the proteasome inhibitor and the therapeutic agent is a subtoxic dose. As used herein, the term subtoxic dose refers to a dose which results in the killing of less than about 10% of the cells.

The regimen of administration can also affect what constitutes an effective amount. The proteasome inhibitor can be administered to the subject prior to, simultaneously with, or after the administration of the therapeutic agent. Further, several divided dosages, as well as staggered dosages, can be administered daily or sequentially, or the dose can be continuously infused. Further, the dosages can be proportionally increased or decreased as indicated by the exigencies of the therapeutic situation.

The term “regimen of administration” refers to the timing of administration and the sequence of administration of the proteasome inhibitor, bendamustine and doxorubicin which allow the combination to perform its intended function of treating multiple myeloma or inhibiting cancer cell growth. The regimen of administration, e.g., the timing and/or sequence of administration, of the proteasome inhibitor, bendamustine and doxorubicin can vary depending on such factors as the pharmacokinetics of the proteasome inhibitor, bendamustine and doxorubicin, the type of cell growth being treated or inhibited, the size of the subject, the severity of the cancer cell growth or tumor, or effective dosage. The choice of each of the individual agents (proteasome inhibitor, bendamustine and doxorubicin) which make up the combination can affect what constitutes a “regimen of administration”. For example, in embodiments where contacting a cell with a proteasome inhibitor after the cell is contacted with a therapeutic agent is desirable, a proteasome inhibitor with slower pharmacokinetics may be administered prior to the administration of the therapeutic agent such that the proteasome inhibitor contacts the cell subsequent to contact with the therapeutic agent. Proteasome inhibitors with rapid pharmacokinetics may be administered after the therapeutic agent such that the proteasome inhibitor contacts the cell subsequent to contact with the therapeutic agent. One of ordinary skill in the art would be able to review the aforementioned factors as well as art known proteasome inhibition studies in determining an appropriate regimen of administration of the proteasome inhibitor and/or therapeutic agent without undue experimentation.

Determination of a therapeutically effective amount of a proteasome inhibitor and a therapeutically effective amount of therapeutic agents, e.g., chemotherapeutic agents, can be readily made by the physician (the “attending clinician”), as one skilled in the art, by the use of known techniques and by observing results obtained under analogous circumstances. The dosages may be varied depending upon the requirements of the patient in the judgment of the attending clinician, the severity of the condition being treated and the particular compound being employed. In determining the therapeutically effective amount or dose, a number of factors are considered by the attending clinician, including, but not limited to: the specific hyperplastic/neoplastic cell involved; pharmacodynamic characteristics of the particular agent and its mode and route of administration; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment; and other relevant circumstances. U.S. Pat. No. 5,427,916, for example, describes method for predicting the effectiveness of antineoplastic therapy in individual patients, and illustrates certain methods which can be used in conjunction with the treatment protocols of the instant invention.

Treatment can be initiated with smaller dosages which are less than the optimum dose of the compound. Thereafter, the dosage should be increased by small increments until the optimum effect under the circumstances is reached. For convenience, the total daily dosage may be divided and administered in portions during the day if desired.

A therapeutically effective amount of a proteasome inhibitor is expected to be from about 1 milligram per square meter of body surface area per day (mg/m² body surface area/day) to about 3 mg/m² body surface area. Depending on the particular inhibitor used, preferred doses of from about 1 mg/m² to about 2 mg/m² body surface area, from about 1 mg/m² to about 1.5 mg/m² body surface area, or from about 2 mg/m² to about 2.5 mg/m² body surface area. Typically, the proteasome inhibitor is administered on specific days in a cycle, in accordance with a regimen of administration. For example, the proteasome inhibitor may be administered on days 1, 4, 8, and 11 of a 21 day cycle. In such a regimen, the foregoing dosages are provided on the administration days, so that the patient is administered, for example, from about 1 mg/m² to about 2 mg/m² body surface area, or from about 1 mg/m² to about 1.5 mg/m² body surface area, etc., on each of days 1, 4, 8 and 11.

A therapeutically effective amount of bendamustine and doxorubicin are well established and/or may be readily determined using art-recognized methods.

The effectiveness of any particular combination of a proteasome inhibitor with bendamustine and doxorubicin to treat multiple myeloma can be monitored by comparing two or more samples obtained from a patient undergoing anti cancer treatment. In general, it is preferable to obtain a first sample from the patient prior to beginning therapy and one or more samples during treatment. In such a use, a baseline of expression of cancer cells prior to therapy is determined and then changes in the baseline state of expression of cancer cells is monitored during the course of therapy. Alternatively, two or more successive samples obtained during treatment can be used without the need of a pre-treatment baseline sample. In such a use, the first sample obtained from the subject is used as a baseline for determining whether the expression of cancer cells is increasing or decreasing.

In general, when monitoring the effectiveness of a therapeutic treatment, two or more samples from the patient are examined. Preferably, three or more successively obtained samples are used, including at least one pretreatment sample.

Provided are methods for treating multiple myeloma in a subject. The subject is administered an effective amount of a combination comprising a proteasome inhibitor, bendamustine and doxorubicin.

In a preferred embodiment, the bendamustine and doxorubicin are administered prior to the administration of the proteasome inhibitor. In another preferred embodiment, the proteasome inhibitor is administered prior to the administration of the bendamustine and doxorubicin.

In a preferred embodiment, the bendamustine and the proteasome inhibitor are administered prior to the administration of doxorubicin. In another preferred embodiment, the doxorubicin is administered prior to the administration of bendamustine and the proteasome inhibitor.

In a preferred embodiment, the proteasome inhibitor and doxorubicin are administered prior to the administration of bendamustine. In another preferred embodiment, the bendamustine is administered prior to the administration of the proteasome inhibitor and doxorubicin.

Also provided is a preferred embodiment where bendamustine is administered, followed by doxorubicin, followed by the proteasome inhibitor. In another preferred embodiment, doxorubicin is administered, followed by bendamustine, followed by the proteasome inhibitor. In another preferred embodiment, the proteasome inhibitor is administered, followed by doxorubicin, followed by bendamustine. In an additional preferred embodiment, the proteasome inhibitor is administered, followed by bendamustine, followed by doxorubicin. In another preferred embodiment, doxorubicin is administered, followed by bendamustine, followed by the proteasome inhibitor. In yet another preferred embodiment, the doxorubicin is administered, followed by the proteasome inhibitor, followed by bendamustine.

Also provided is a preferred embodiment where the bendamustine is administered on days 4 and 5 of a 21-day cycle, the proteasome inhibitor is administered on days 1, 4, 8 and 11 of a 21-day cycle, and the doxorubicin is administered on day 4 of a 21-day cycle. Preclinical models have shown that recovery of proteasome activity was nearly complete by 48-72 hours following bortezomib dosing. Kane et al, “Velcade®: U.S. FDA approval for the treatment of multiple myeloma progressing on prior therapy.” The Oncologist(2003) 8:508-513. The waiting periods built in to the 21 day cycle thus minimize toxicity. Richardson et al, “Bortezomib: proteasome inhibition as an effective anticancer therapy.” Annual Review of Medicine (2006) 57:33-47. According to the patient safety information provided on the VELCADE® website, VELCADE® is associated with thrombocytopenia and neutropenia that follow a cyclical pattern with nadirs occurring following the last dose of each cycle and typically recovering prior to initiation of the subsequent cycle. (See, e.g., http://www.velcade.com/pat_safety_info_mcl.html). The cyclical pattern of platelet and neutrophil decreases and recovery remained consistent over the 8 cycles of twice weekly dosing, and there was no evidence of cumulative thrombocytopenia or neutropenia.

In a further preferred embodiment a method is provided according to any one of the preceding embodiments, wherein the bendamustine is administered at a dose of from about 75 to about 250 mg/m²/day.

In another preferred embodiment, a method is provided according to any one of the preceding embodiments, wherein the proteasome inhibitor is bortezomib and is administered at a dose of from about 1.0 to about 1.5 mg/m². Dosages for other proteasome inhibitors will depend on the inhibitor selected. For example, a preferred dose for [(1R)-1-[[(2S,3R)-3-hydroxy-2-[6-phenyl-pyridine-2-carbonyl)amino]-1-oxobutyl]amino]-3-methylbutylboronic acid may be from about 2.0 to about 2.5 mg/m², as described previously.

In another preferred embodiment, a method is provided according to any one of the preceding embodiments, wherein the doxorubicin is in the form of liposomal doxorubicin, administered at a dose of from about 20 to about 40 mg/m², preferably about 30 mg/m².

In another preferred embodiment, a method is provided according to any one of the preceding embodiments, wherein the bendamustine is administered at a dose of about 90 mg/m²/day, the proteasome inhibitor is administered at a dose of about 1.3 mg/m² and the doxorubicin is administered in the form of liposomal doxorubicin at a dose of about 30 mg/m².

In certain aspects of the invention, the bendamustine is administered at a dose of about 120 mg/m²/day, a proteasome inhibitor is administered at a dose of about 1.3 mg/m² and the doxorubicin is administered in the form of liposomal doxorubicin at a dose of about 30 mg/m².

In other aspects of the invention, the bendamustine is administered at a dose of about 150 mg/m²/day, a proteasome inhibitor is administered at a dose of about 1.3 mg/m² and the doxorubicin is administered in the form of liposomal doxorubicin at a dose of about 30 mg/m².

In further aspects of the invention, the bendamustine is administered at a dose of about 180 mg/m²/day, a proteasome inhibitor is administered at a dose of about 1.3 mg/m² and the doxorubicin is administered in the form of liposomal doxorubicin at a dose of about 30 mg/m².

In further aspects of the invention, the bendamustine is administered at a dose of about 210 mg/m²/day, a proteasome inhibitor is administered at a dose of about 1.3 mg/m² and the doxorubicin is administered in the form of liposomal doxorubicin at a dose of about 30 mg/m².

In another aspect of the invention, a method is provided according to any one of the preceding embodiments, further comprising administering a granulocyte colony-stimulating factor (G-CSF) such as filgrastim. In a preferred embodiment, filgrastim is administered at a dose of about 5 μg/kg/day SC starting day 6 until neutrophil recovery to ANC>1000. ANC is an abbreviation for “absolute neutrophil count.”

In preferred embodiments, the proteasome inhibitor, doxorubicin and/or bendamustine are administered intravenously. In other preferred embodiments, the proteasome inhibitor, doxorubicin and/or bendamustine are administered intraperitoneally. In further preferred embodiments, the proteasome inhibitor, doxorubicin and/or bendamustine are administered orally.

In preferred embodiments, the proteasome inhibitor is bortezomib.

In further preferred embodiments, the proteasome inhibitor is [(1R)-1-[[(2S,3R)-3-hydroxy-2-[6-phenyl-pyridine-2-carbonyl)amino]-1-oxobutyl]amino]-3-methylbutylboronic acid.

When the proteasome inhibitors, bendamustine and doxorubicin of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

Combination treatments involving a proteasome inhibitor, doxorubicin and bendamustine can be achieved by contacting cells with the proteasome inhibitor, bendamustine and doxorubicin at the same time. Such combination treatment can be achieved by contacting the cell with a single composition or pharmaceutical formulation that includes the proteasome inhibitor, bendamustine and doxorubicin, or by contacting the cell with two or more distinct compositions or formulations, at the same time, wherein one composition includes the proteasome inhibitor, another composition includes bendamustine or doxorubicin, and another composition includes bendamustine or doxorubicin.

Alternatively, administration of the proteasome inhibitor can precede or follow treatment with either bendamustine or doxorubicin by intervals ranging from minutes to weeks. In embodiments where the bendamustine, doxorubicin and the proteasome inhibitor are applied separately to the cell, one would generally ensure that a significant period of time did not expire between the time of each delivery, such that the proteasome inhibitor, bendamustine and doxorubicin would still be able to exert an advantageously combined effect on the cell. In such instances, it is provided that one would contact the cell with each modality within about 12-24 hours of each other and, optionally, within about 6-12 hours of each other. In some situations, it can be desirable to extend the time period for treatment significantly, however, where several days (2, 3, 4, 5, 6 or 7) to several weeks (1, 2, 3, 4, 5, 6, 7 or 8) lapse between the respective administrations. Also, under some circumstances, more than one administration of any of the proteasome inhibitor, bendamustine or doxorubicin will be desired.

Additional cancer treatments can be used in combination with the administration of the proteasome inhibitor, bendamustine and doxorubicin. For example, the combination therapy of a proteasome inhibitor, bendamustine and doxorubicin may be used as part of a treatment course further involving attempts to surgically remove part or all of a cancerous growth. For instance, the combination therapy may be administered after surgical treatment of a subject to treat any remaining neoplastic or metastasized cells. Treatment can also precede surgery, in an effort to shrink the size of the tumor to reduce the amount of tissue to be excised, thereby making the surgery less invasive and traumatic.

Treating multiple myeloma with the combination therapy of the presently disclosed subject matter can further include one or more treatment courses with a radiotherapeutic agent to induce DNA damage. Radiotherapeutic agents include, for example, gamma irradiation, X-rays, UV-irradiation, microwaves, electronic emissions, radioisotopes and the like. Therapy can be achieved by irradiating the localized tumor site with the above-described forms of radiation.

Another aspect of the invention relates to methods for purging bone marrow, i.e., removing cancer cells from bone marrow, by exposing the bone marrow cells to the proteasome inhibitor, doxorubicin and bendamustine combination therapy. The purged bone marrow may then be placed back into the subject from which the bone marrow was removed, or placed into a different subject.

### Example 1

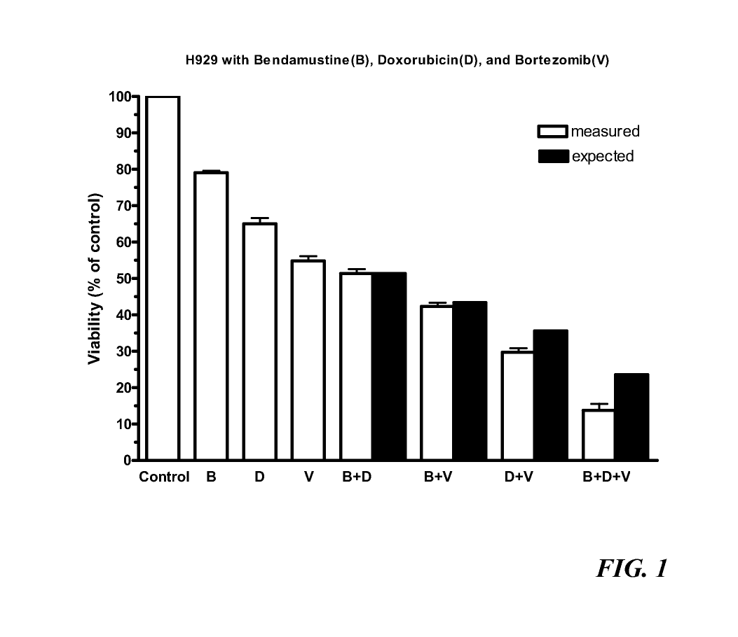

H929 and MM1R myeloma cell lines were cultured in vitro and cellular cytotoxicity was measured by MTS ([3-(4,5-dimethylthiazol-2-yl)-5-(3-carboxymethoxyphenyl)-2-(4-sulfophenyl)-2H-tetrazolium, inner salt) assay. The effect of the combination in terms of additive, antagonistic, or synergistic activity was assessed by Valeriote and Lin's method (Valeriote and Lin, Cancer Chemother Rep 1975; 59:895-900). In repeated experiments, bendamustine alone induced MM cell death in a time and dose-dependent manner, with a bendamustine concentration that was lethal to 50% of cells (LC₅₀) after 48 hours of 34.9±3.4 μg/ml. Further, when combined with doxorubicin, bendamustine showed no antagonistic effect when cells were exposed to drug for 48 hours. The measured cell survival for the combination of B+D was similar to the calculated expected cell survival based on individual drug activity, indicating an additive effect (FIG. 1). In addition, bendamustine showed an additive effect when combined with bortezomib, and as previously shown doxorubicin and bortezomib were synergistic (FIG. 1). To test the effect of the combination of all three drugs, cells were cultured with bendamustine (15 μg/ml) and doxorubicin (0.03 μg/ml) for 48 hours, and bortezomib (2.5 nmol/L) was added in the last 24 hours, and cytotoxicity was assayed. The expected cell survival for the combination of B+(D+V) is lower than the product of the effects of B and D+V (FIG. 1), indicating a synergistic effect of bendamustine when combined with doxorubicin and bortezomib.

Accordingly, a first aspect of the invention provides a method for treating multiple myeloma in a subject comprising administering to the subject an effective amount of a combination comprising a proteasome inhibitor, bendamustine and doxorubicin.

A second aspect of the invention provides a method according to the first aspect, wherein the bendamustine and doxorubicin are administered prior to administration of the proteasome inhibitor

A third aspect of the invention provides a method according to the first aspect, wherein the proteasome inhibitor is administered prior to the administration of the bendamustine and doxorubicin.

A fourth aspect of the invention provides a method according to the first aspect, wherein the bendamustine and the proteasome inhibitor are administered prior to the administration of doxorubicin.

A fifth aspect of the invention provides a method according to the first aspect, wherein the doxorubicin is administered prior to the administration of bendamustine and the proteasome inhibitor.

A sixth aspect of the invention provides a method according to the first aspect, wherein the proteasome inhibitor and doxorubicin are administered prior to the administration of bendamustine.

A seventh aspect of the invention provides a method according to the first aspect, wherein the bendamustine is administered prior to the administration of the proteasome inhibitor and doxorubicin.

An eighth aspect of the invention provides a method according to the first aspect, wherein bendamustine is administered, followed by doxorubicin, followed by the proteasome inhibitor.

A ninth aspect of the invention provides a method according to the first aspect, wherein doxorubicin is administered, followed by bendamustine, followed by the proteasome inhibitor.

A tenth aspect of the invention provides a method according to the first aspect, wherein the proteasome inhibitor is administered, followed by doxorubicin, followed by bendamustine.

An eleventh aspect of the invention provides a method according to the first aspect, wherein the proteasome inhibitor is administered, followed by bendamustine, followed by doxorubicin.

A twelfth aspect of the invention provides a method according to the first aspect, wherein the doxorubicin is administered, followed by bendamustine, followed by the proteasome inhibitor.

A thirteenth aspect of the invention provides a method according to the first aspect, wherein the doxorubicin is administered, followed by the proteasome inhibitor, followed by bendamustine.

A fourteenth aspect of the invention provides a method according to the first aspect, wherein the bendamustine is administered on days 4 and 5 of a 21-day cycle;

the proteasome inhibitor is administered on days 1, 4, 8 and 11 of a 21-day cycle; and

the doxorubicin is administered on day 4 of a 21-day cycle.

A fifteenth aspect of the invention provides a method according to any one of the first through fourteenth aspects, wherein the bendamustine is administered at a dose of from about 75 to about 250 mg/m²/day.

A sixteenth aspect of the invention provides a method according to any one of the first through fifteenth aspects, wherein the proteasome inhibitor is administered at a dose of from about 1.0 to about 1.5 mg/m².

A seventeenth aspect of the invention provides a method according to any one of the first through fifteenth aspects, wherein the proteasome inhibitor is administered at a dose of from about 2.0 to about 2.5 mg/m².

An eighteenth aspect of the invention provides a method according to any one of the first through seventeenth aspects, wherein the doxorubicin is in the form of liposomal doxorubicin, administered at a dose of from about 20 to about 40 mg/m².

A nineteenth aspect of the invention provides a method according to any one of the first through eighteenth aspects wherein the bendamustine is administered at a dose of about 90 mg/m²/day, the proteasome inhibitor is administered at a dose of about 1.3 mg/m² and the doxorubicin is administered in the form of liposomal doxorubicin at a dose of about 30 mg/m².

A twentieth aspect of the invention provides a method according to any one of the first through eighteenth aspects wherein the bendamustine is administered at a dose of about 120 mg/m²/day, a proteasome inhibitor is administered at a dose of about 1.3 mg/m² and the doxorubicin is administered in the form of liposomal doxorubicin at a dose of about 30 mg/m².

A twenty-first aspect of the invention provides a method according to any one of the first through eighteenth aspects wherein the bendamustine is administered at a dose of about 150 mg/m²/day, a proteasome inhibitor is administered at a dose of about 1.3 mg/m² and the doxorubicin is administered in the form of liposomal doxorubicin at a dose of about 30 mg/m².

A twenty-second aspect of the invention provides a method according to any one of the first through eighteenth aspects wherein the bendamustine is administered at a dose of about 180 mg/m²/day, a proteasome inhibitor is administered at a dose of about 1.3 mg/m² and the doxorubicin is administered in the form of liposomal doxorubicin at a dose of about 30 mg/m².

A twenty-third aspect of the invention provides a method according to any one of the first through eighteenth aspects wherein the bendamustine is administered at a dose of about 210 mg/m²/day, a proteasome inhibitor is administered at a dose of about 1.3 mg/m² and the doxorubicin is administered in the form of liposomal doxorubicin at a dose of about 30 mg/m².

A twenty-fourth aspect of the invention provides a method according to any one of the first through twenty-third aspects, further comprising administering filgrastim.

A twenty-fifth aspect of the invention provides a method according to the twenty-fourth aspect wherein said filgrastim is administered at a dose of about 5 μg/kg/day SC starting day 6 until neutrophil recovery to ANC>1000.

A twenty-sixth aspect of the invention provides a method according to any one of the first through twenty-fifth aspects wherein the proteasome inhibitor, doxorubicin and bendamustine are administered intravenously.

A twenty-seventh aspect of the invention provides a method according to any one of the first through twenty-fifth aspects wherein the proteasome inhibitor, doxorubicin and bendamustine are administered intraperitoneally.

A twenty-eighth aspect of the invention provides a method according to any one of the first through twenty-fifth aspects wherein the proteasome inhibitor, doxorubicin and bendamustine are administered orally.

A twenty-ninth aspect of the invention provides a method according to any one of the first through twenty-eighth aspects wherein the proteasome inhibitor is bortezomib.

A thirtieth aspect of the invention provides a method according to any one of the first through twenty-eighth aspects wherein the proteasome inhibitor is [(1R)-1-[[(2S,3R)-hydroxy-2-[6-phenyl-pyridine-2-carbonyl)amino]-1-oxobutyl]amino]-3-methylbutylboronic acid.

What is claimed:

1. A method for treating multiple myeloma in a subject comprising administering to the subject an effective amount of a combination comprising a proteasome inhibitor, bendamustine and doxorubicin.

2. The method according to claim 1, wherein the bendamustine and doxorubicin are administered prior to administration of the proteasome inhibitor

3. The method according to claim 1, wherein the proteasome inhibitor is administered prior to the administration of the bendamustine and doxorubicin.

4. The method according to claim 1, wherein the bendamustine and the proteasome inhibitor are administered prior to the administration of doxorubicin.

5. The method according to claim 1, wherein the doxorubicin is administered prior to the administration of bendamustine and the proteasome inhibitor.

6. The method according to claim 1, wherein the proteasome inhibitor and doxorubicin are administered prior to the administration of bendamustine.

7. The method according to claim 1, wherein the bendamustine is administered prior to the administration of the proteasome inhibitor and doxorubicin.

8. The method according to claim 1, wherein bendamustine is administered, followed by doxorubicin, followed by the proteasome inhibitor.

9. The method according to claim 1, wherein doxorubicin is administered, followed by bendamustine, followed by the proteasome inhibitor.

10. The method according to claim 1, wherein the proteasome inhibitor is administered, followed by doxorubicin, followed by bendamustine.

11. The method according to claim 1, wherein the proteasome inhibitor is administered, followed by bendamustine, followed by doxorubicin.

12. The method according to claim 1, wherein the doxorubicin is administered, followed by bendamustine, followed by the proteasome inhibitor.

13. The method according to claim 1, wherein the doxorubicin is administered, followed by the proteasome inhibitor, followed by bendamustine.

14. The method according to claim 1, wherein the bendamustine is administered on days 4 and 5 of a 21-day cycle;
the proteasome inhibitor is administered on days 1, 4, 8 and 11 of a 21-day cycle; and
the doxorubicin is administered on day 4 of a 21-day cycle.

15. The method according to claim 1, wherein the bendamustine is administered at a dose of from about 75 to about 250 mg/m²/day.

16. The method according to claim 1, wherein the proteasome inhibitor is administered at a dose of from about 1.0 to about 1.5 mg/m².

17. The method according to claim 1, wherein the proteasome inhibitor is administered at a dose of from about 2.0 to about 2.5 mg/m².

18. The method according to claim 1, wherein the doxorubicin is in the form of liposomal doxorubicin, administered at a dose of from about 20 to about 40 mg/m².

19. The method according to claim 1 wherein the bendamustine is administered at a dose of about 90 mg/m²/day, the proteasome inhibitor is administered at a dose of about 1.3 mg/m² and the doxorubicin is administered in the form of liposomal doxorubicin at a dose of about 30 mg/m².

20. The method according to claim 1 wherein the bendamustine is administered at a dose of about 120 mg/m²/day, a proteasome inhibitor is administered at a dose of about 1.3 mg/m² and the doxorubicin is administered in the form of liposomal doxorubicin at a dose of about 30 mg/m².

21. The method according to claim 1 wherein the bendamustine is administered at a dose of about 150 mg/m²/day, a proteasome inhibitor is administered at a dose of about 1.3 mg/m² and the doxorubicin is administered in the form of liposomal doxorubicin at a dose of about 30 mg/m².

22. The method according to claim 1 wherein the bendamustine is administered at a dose of about 180 mg/m²/day, a proteasome inhibitor is administered at a dose of about 1.3 mg/m² and the doxorubicin is administered in the form of liposomal doxorubicin at a dose of about 30 mg/m².

23. The method according to claim 1 wherein the bendamustine is administered at a dose of about 210 mg/m²/day, a proteasome inhibitor is administered at a dose of about 1.3 mg/m² and the doxorubicin is administered in the form of liposomal doxorubicin at a dose of about 30 mg/m².

24. The method according to claim 1, further comprising administering filgrastim.

25. The method of claim 24 wherein said filgrastim is administered at a dose of about 5 μg/kg/day SC starting day 6 until neutrophil recovery to ANC>1000.

26. The method according to claim 1 wherein the proteasome inhibitor, doxorubicin and bendamustine are administered intravenously.

27. The method according to claim 1 wherein the proteasome inhibitor, doxorubicin and bendamustine are administered intraperitoneally.

28. The method according to claim 1 wherein the proteasome inhibitor, doxorubicin and bendamustine are administered orally.

29. The method according to claim 1 wherein the proteasome inhibitor is bortezomib.

30. The method according to claim 1 wherein the proteasome inhibitor is [(1R)-1-[[(2S,3R)-3-hydroxy-2-[6-phenyl-pyridine-2-carbonyl)amino]-1-oxobutyl]amino]-3-methylbutylboronic acid.
